(19) United States
(12) Patent Application Publication
(10) Pub. No.: US 2020/0023033 A1
(43) Pub. Date: Jan. 23, 2020

(54) COMPOSITION FOR TREATING PULMONARY FIBROSIS COMPRISING ALLOFERON

(71) Applicant: AT-Pharma Co., Ltd., Seongnam (KR)

(72) Inventors: Jae Seung Kang, Seongnam (KR); Wang Jae Lee, Seongnam (KR)

(21) Appl. No.: 16/514,849

(22) Filed: Jul. 17, 2019

Related U.S. Application Data
(60) Provisional application No. 62/699,179, filed on Jul. 17, 2018.

(51) Int. Cl. A61K 38/08; A61P 11/00

(52) U.S. Cl. CPC A61K 38/08; A61P 11/00

(57) ABSTRACT

It has been found that the compounds of formula (I) provide unexpected advantages in the treatment of pulmonary fibrosis. In particular, methods of treating pulmonary fibrosis using compounds of formula (I) is disclosed.

### PRIORITY

This application claims the benefit of priority based upon U.S. Provisional Patent Application having Application No. 62/699,179, filed on Jul. 17, 2018, and entitled “ADJUVANT THERAPY WITH THE USE OF ALLOFERON,” which is hereby incorporated herein by reference in its entirety.

### BACKGROUND

### Field

The present disclosure generally relates to the use of peptides (alloferons) of formula (I), X₁-His-Gly-X₂₋His-Gly-Val-X₃, wherein the groups X₁ to X₃ have the meanings given in the claims and specification or a pharmaceutically acceptable salt thereof, in the treatment of pulmonary fibrosis.

### Related Art

The compounds of formula (I) have been shown to immunomodulate anticancer activity in animal. In particular, an alloferon has been shown to stimulate the natural cytotoxicity of human peripheral blood lymphocytes, induce interferon synthesis in mouse and human models, and enhance antiviral and antitumor resistance in mice. (Chernysh, Proceedings of the National Academy of Science of the United States 99, 12628 (2002)). It has been suggested that alloferons have a therapeutic effect similar to interferons, but the chemical structure of alloferon do not share any similarity with interferons, other known cytokines and interferon inducers as well as any other materials of medical importance. (U.S. Pat. No. 6,692,747).

Alloferons have been shown to enhance natural killer (NK) cell cytotoxicity. (Bae, Immunobiology 218(8), 1026 (2013)). NK cells are re a type of lymphocyte and a component of innate immune system. NK cells are known to play a key role in antitumor and antiviral immunity. Bae suggests that alloferon has antitumor effects through up-regulation of NK-activating receptor 2B4 and the enhancement of granule exocytosis from NK cells.

Peptides of formula (I), X₁-His-Gly-X₂₋His-Gly-Val-X₃, wherein X₁ is absent or represents at least one amino acid residue, X2 is a peptide bond or represents at least one amino acid residue, and X₃ is absent or represents at least one amino acid residue are disclosed in U.S. Pat. Nos. 6,692,747 and 7,462,360 as biologically active peptides specifically stimulating antiviral, antimicrobial, and antitumor activity of the human and animal immune system. These patents also disclose methods of manufacturing a composition having immunomodulatory activity, comprising combining a peptide of formula (I). The disclosure of U.S. Pat. Nos. 6,692,747 and 7,462,360 and WO 2005/037,824 are incorporated by reference.

Pulmonary fibrosis is a condition characterized by a chronic and progressive scarring and stiffening of the air sac in the lungs (alveoli) making it difficult to breathe and get enough oxygen into the bloodstream. Pulmonary fibrosis can be caused by several different conditions, including inhalation of certain materials, such as silica dust, asbestos fibers, hard metal dusts, coal dust, grain dust, and bird and animal droppings. Many medical conditions associated with lung inflammation can also lead to pulmonary fibrosis, including pneumonia, dermatomyositis, polymyositis, sarcoidosis, systemic lupus erythematosus, mixed connective tissue disease, rheumatoid arthritis, and scleroderma.

The diagnosis of pulmonary fibrosis can usually be made by careful history, including exposure to certain materials, physical examination, chest radiography, including a high-resolution computer tomographic scan (HRCT), and open lung or transbronchial biopsies. In up to two-thirds of the patients exhibiting symptoms of pulmonary fibrosis, no underlying cause for the pulmonary fibrosis can be found. These conditions of unknown etiology have been termed idiopathic interstitial pneumonias. Histologic examination of tissue obtained at open lung biopsy allows classification of these patients into several categories, including Usual Interstitial Pneumonia (UIP), Desquamative Interstitial Pneumonia (DIP), and Non-Specific Interstitial Pneumonia (NSIP).

Pulmonary fibrosis involves the overgrowth, hardening, and scarring of lung tissue due to excess deposition by fibroblasts of extracellular matrix components, such as collagen. Fibroblasts serve roles in inflammation and immune cell recruitment to sites of tissue injury. Furthermore, fibroblasts produce and are responsive to many inflammatory cytokines. Fibroblasts thus can contribute to chronic inflammation, and reciprocally, inflammatory cytokines promote fibroblast to myofibroblast transition, facilitating fibrosis. Thus, injury or inflammation of lung tissue can lead to pulmonary fibrosis.

Pirfenidone (Esbriet®) and nintedanib (Ofev®) have been approved by the U.S. F.D.A. for the treatment of pulmonary fibrosis. For pirfenidone, the recommended treatment regimen is to begin a patient on one 267 mg tablet per day for the first week (days 1-7 of treatment). On day 8, the dosage is increased to two 267 mg tablets per day for the second week (days 8-14 of treatment). On day 15, the dosage is increased to three 267 mg tablets per day. After the full dose of three 267 mg tablets or capsules TID is well tolerated, the patient is transitioned to one 801 mg tablet per day for a maintenance option with fewer pills per day. The recommended dosage of nintedanib is 150 mg twice a day with the dosages given approximately twelve hours apart.

At present, there is no treatment that can reverse or stop the progression of pulmonary fibrosis. Recently approved therapeutics, such as pirfenidone (Esbriet®) and nintedanib (Ofev®) merely slow down the progression of pulmonary fibrosis and are associated with several serious side effects. Thus, a need exists for a safe and effective therapeutic regimen to stop the progression of pulmonary fibrosis.

### SUMMARY

It has been found that the compounds of formula (I) provide unexpected advantages in the treatment of pulmonary fibrosis. Compounds of formula (I) have been found to greatly reduce the inflammation associated with pulmonary fibrosis. Moreover, treatment with compounds of formula (I) also completely suppressed the deposition of collagen in fibrotic tissue.

A first aspect of the present invention therefore is a composition for treating pulmonary fibrosis, said composition comprising a compound of formula (I).

A second aspect of the present invention therefore is a method of treating pulmonary fibrosis, said method comprising administering a therapeutically effective amount of a compound of formula (I) as a monotherapy to a patient in need thereof.

### BRIEF DESCRIPTION OF THE DRAWINGS

The patent or application file contains at least one drawing executed in color.

Copies of this patent or patent application publication with color drawings(s) will be provided by the Office upon request and payment of the necessary fee.

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 illustrates histology of lung tissue stained to show collagen deposition.

FIG. 2 illustrates histology of lung tissue stained to show collagen deposition after treatment with alloferon.

FIG. 3 shows lung tissue stained for collagen from (a) a control animal, (b) an animal treated with bleomycin, and (c) an animal treated with bleomycin that was also treated with alloferon.

FIG. 4 shows a table with a measurement of collagen content in lung tissue from (a) a control animal, (b) an animal treated with bleomycin, and (c) an animal treated with bleomycin that was also treated with alloferon.

FIG. 5 illustrates bronchoalveolar lavage fluid cell analysis for tissue from bleomycin-induced pulmonary fibrosis.

FIG. 6 illustrates analysis for inflammatory cell population in bronchoalveolar lavage fluid by alloferon treatment

FIG. 7 shows significant increase of IL-17+ and IFN-γ⁺ cells in alloferon injected group compared to PBS-treated control group

FIG. 8 shows significant decrease of IL-6, TNF-α, and MCP-1, MIP-1, and MIP-2 in alloferon injected group compared to PBS-treated control group

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure.

In a first embodiment (1), both with regard to the first and second aspect of the invention, formula (I)

X₁-His-Gly-X₂₋His-Gly-Val-X₃ (I)

is defined to encompass those compounds wherein
X₁ is absent or represents at least one amino acid residue,
X₂ is a peptide bound or represents at least one amino acid residue, and
X₃ is absent or represents at least one amino acid residue, or a pharmaceutically acceptable salt or ether thereof, the peptide exhibiting immunomodulatory activity.

In describing one embodiment, the compounds of the above formula (1) is referred to as the ‘alloferon’. In a preferred embodiment, the alloferon has the amino acid sequence of SEQ ID NO:1 or SEQ ID NO:2. In an especially preferred embodiment, the alloferon has the sequence of SEQ ID NO:1. Alloferons may be obtained by separating and refining from nature or by synthesizing it.

The peptides of formula (I) may be synthesized in any conventional method known in the art. For example, synthesis can be performed using solid-phase peptides synthesis using Boc/Bzl strategies of phenyl acetamide methyl polymer (PAM). (WO2013/176563). Purification of the peptides of the present invention may be performed, for example, using a two-step protocol. The first step was performed on Sep-Pak Vac columns with C18 sorbent (Waters) by means of the column elution by 40% acetonitrile acidified by 0.05% trifluoroacetic acid. Next, the peptide was purified to homogeneity using preparatory HPLC equipped with an C18 column in the linear gradient of 0.05% trifluoroacetic acid and acidified acetonitrile (0-20% acetonitrile during 40 min under flow rate 2.5 ml/min and detector wave length 225 nm). The peptide purity was confirmed by MALDI-TOF mass spectrometry, and the amino acid sequence of the peptide was checked by microsequencing.

In addition to the peptides described herein, peptidomimetics are also contemplated. Peptide analogs are commonly used in the pharmaceutical industry as non-peptide drugs with properties analogous to those of the template peptide. Such peptidomimetics include chemically modified peptides, peptide- like molecules containing non-naturally occurring amino acids, and peptoids. These types of non-peptide compound are termed “peptide mimetics” or “peptidomimetics” (Evans, J. Med. Chem. 30, 1229 (1987)) and are usually developed with the aid of computerized molecular modeling. Peptide mimetics that are structurally similar to therapeutically useful peptides may be used to produce an equivalent therapeutic or prophylactic effect.

The peptide or peptidomimetic may be cyclic or otherwise conformationally constrained. Conformationally constrained molecules can have improved properties such as increased affinity, metabolic stability, membrane permeability or solubility. Methods of conformational constraint are well known in the art.

Compositions comprising compounds of formula (I) may be prepared. The composition according to the invention for parenteral administration is generally in the form of a solution or suspension of the peptide in a pharmaceutically acceptable carrier, preferably an aqueous carrier. Examples of aqueous carriers that may be used include water, buffered water, saline solution (0.4%), glycine solution (0.3%), hyaluronic acid and similar known carriers. Apart from aqueous carriers it is also possible to use solvents such as dimethylsulphoxide, propyleneglycol, dimethylformamide and mixtures thereof. The composition may also contain pharmaceutically acceptable excipients such as buffer substances and inorganic salts in order to achieve normal osmotic pressure and/or effective lyophilization. Examples of such additives are sodium and potassium salts, e.g., chlorides and phosphates, sucrose, glucose, protein hydrolysates, dextran, polyvinylpyrrolidone or polyethylene glycol. The compositions may be sterilized by conventional methods, e.g., by sterile filtration. The composition may be decanted directly in this form or lyophilized and mixed with a sterile solution before use. The compositions may be formulated into various forms suitable for oral or non-oral administration. The compositions may be formulated according to any of the methods known in the art.

In a preferred embodiment, the invention relates to the use of a compound of formula (I) according to the invention as a monotherapy to treat pulmonary fibrosis.

Depending on the type and severity of the disease, about 1 μg/kg to 1000 mg/kg of body weight once per day of the compound of formula (I) is an initial candidate dosage for administration to the patient, whether, for example, by one or more separate administrations, or by continuous infusion. A typical daily dosage might range from about 1 μg/kg to 100 mg/kg or more, depending on the factors mentioned above, more preferably about 0.1 to 20 mg/kg of body weight, and, when administered subcutaneously or intraperitoneally, about 1 to 20 mg/kg of body weight. Necessary modifications in this dosage range may be determined by one of ordinary skill in the art using only routine experimentation given the teachings herein. (Remington's Pharmaceutical Sciences, 20th edition, (ed. A. Gennaro; Lippincott, Williams & Wilkins 2000)). For repeated administrations over several days or longer, depending on the condition, the treatment is sustained until a desired suppression of disease symptoms occurs. However, other dosage regimens may be useful. The progress of the therapy according to the invention is easily monitored by conventional techniques and assays.

Compositions comprising compounds of formula (I) may be administered in a number of ways. Representative delivery regimens include oral, parenteral (including subcutaneous, intramuscular and intravenous injection), rectal, buccal (including sublingual), transdermal, inhalation ocular and intranasal. In one embodiment, delivery of peptides entails subcutaneous injection of a controlled-release injectable formulation. In some embodiments, peptides and/or proteins described herein are useful for subcutaneous, intranasal and inhalation administration.

In order to assist in understanding the invention, the invention is described in more detail in accordance with the example below. However, these examples only illustrate this invention and do not limit the scope of the claims attached, and it is clear that various changes and modifications can be made within the categories of this invention and within the scope of technical thought, and these changes and modifications are within the scope of the claims attached.

### EXAMPLE 1

### Materials and Methods

Intratracheal inoculation of bleomycin. Eight to ten-week-old male C57BL/6 mice weighing 24 to 28 g were used for the experiments. After measuring their body weight, the mice were anesthetized with an intraperitoneal injection of avertin (Sigma Aldrich). The trachea of the mice was exposed by a 1.0 cm longitudinal incision in the neck and injected with 55 μl of a bleomycin hydrochloride (Nippon Kayaku Co., Tokyo, Japan) solution containing 1.5 mg or 2.0 mg of bleomycin dissolved in a sterile phosphate-buffered saline solution per kilogram of body weight. The test mice were treated with 50 μg alloferon intraperitoneally daily from the day of bleomycin inoculation. All procedures were conducted in a sterile environment and were reviewed and approved by Ethics Committee of the Seoul National University.

Histopathological scoring. Mice were euthanized with CO₂ asphyxiation. After thoracotomy, the lungs were perfused with saline via the right ventricle and inflated with 2 ml of phosphate-buffered 4% paraformaldehyde solution via the trachea and fixed for 24 hours. Routine light microscopic techniques were done for paraffin embedding and the sections were stained with H&E and Masson's trichrome

Bronchoalveolar (BAL) cell counting. Mice were sacrificed by CO₂ asphyxiation in a CO₂ chamber. The mice were dampened with 70% ethanol in a biosafety cabinet. The mice were then placed front side up on a Styrofoam panel, and the arms and legs of the mice were fixed with needles or tape. Scissors were used to make an incision in the skin from abdomen to neck, and the skin retracted with forceps to expose the thoracic cage and neck. The muscle around the neck was gently removed to expose the trachea. Forceps were used to put an approximately 10 cm-long nylon string under the trachea. The ribs were then cut to expose the heart and the lungs without cutting the trachea and lungs. A 22G×1 in. Exel Safelet Catheter was inserted into the trachea, the stylet hub removed, and the catheter and the trachea were tied together firmly with the nylon string. A 1 ml syringe was loaded with 0.8 ml of phosphate-buffered saline (PBS) and placed at the end of the catheter. The PBS was injected and aspirated four times. The syringe was then removed from the catheter, and the recovered lavage fluid saved in 1.5 ml Eppendorf tubes on ice. The BAL volume was recorded according to the scales on the 1.5 ml Eppendorf tubes. The BAL fluid was centrifuged at 800×g for 10 min at 4° C. After centrifugation, the supernatant was transferred to a new 5 ml tube, with a protease inhibitor cocktail added to a final concentration of 1× and PMSF to a final concentration of 1 mM and mixed well. The BAL cell pellets were resuspended in 400 μl of PBS, and the cells counted by taking about 20 μl of the cell sample to a hemocytometer and counting the cells under a microscope.

Collagen content measurement. Lung tissue was homogenized in 100 μl ddH₂O. To a 100 μl of sample homogenate, 100 μl concentrated HCl (˜12 M) was added in a pressure-tight Teflon capped vial. The samples were hydrolyzed at 120° C. for 3 hrs. After homogenization, the samples were clarified with activated charcoal by adding 4 mg of activated charcoal. The samples were then vortexed and centrifuged at 10000×g for three min to remove the precipitate and activated charcoal. 10-30 μl of each hydrolyzed sample was transferred to a 96-well plate and evaporated to dryness under vacuum/on a hot plate/in an oven. A 1.0 mg/ml Collagen I Standard was prepared by adding 50 μl of 2 mg/ml Type I Standard to 50 μl of 0.02 M acetic acid and used to generate 0, 2, 4, 6, 8 and 10 μg of collagen/well. The volume was adjusted to 10 μ/vial with 0.02 M Acetic Acid. 10 μl of 12 M HCl was then added to the pressure-tight Teflon capped vial and hydrolyzed at 120° C. for 3 hrs. The vials were placed on ice, and the contents spun down. The contents of each vial (˜15 μl) were transferred to a 96-well plate and evaporated to dryness under vacuum/on a hot plate/in an oven. 100 μl of the Chloramine T reagent was added to each sample and standard and incubates at room temperature for 5 min 100 μl of the DMAB reagent was then added to each well and incubated for 90 min. at 60° C. Absorbance was measured at 560 nm in a microplate reader. Total Collagen Concentration (C) was calculated as follows (C)=B/V×D μg/μl (B; amount of Collagen in the sample well from Standard Curve (μg), V; sample volume added into the reaction well (μl), D: sample dilution factor).

Inflammatory cell population analysis in BAL. Mice were sacrificed by CO₂ asphyxiation in a CO₂ chamber. The mice were dampened with 70% ethanol in a biosafety cabinet. The mice were then placed front side up on a Styrofoam panel, and the arms and legs of the mice were fixed with needles or tape. Scissors were used to make an incision in the skin from abdomen to neck, and the skin retracted with forceps to expose the thoracic cage and neck. The muscle around the neck was gently removed to expose the trachea. Forceps were used to put an approximately 10 cm-long nylon string under the trachea. The ribs were then cut to expose the heart and the lungs without cutting the trachea and lungs. A 22G×1 in. Exel Safelet Catheter was inserted into the trachea, the stylet hub removed, and the catheter and the trachea were tied together firmly with the nylon string. A 1 ml syringe was loaded with 0.8 ml of phosphate-buffered saline (PBS) and placed at the end of the catheter. The PBS was injected and aspirated four times. The syringe was then removed from the catheter, and the recovered lavage fluid saved in 1.5 ml Eppendorf tubes on ice. The BAL volume was recorded according to the scales on the 1.5 ml Eppendorf tubes. The BAL fluid was centrifuged at 800×g for 10 mM at 4° C. After centrifugation, the supernatant was transferred to a new 5 ml tube, with a protease inhibitor cocktail added to a final concentration of 1× and PMSF to a final concentration of 1 mM and mixed well. The BAL cell pellets were resuspended in 400 μl of PBS, and the cells counted by taking about 20 μl of the cell sample to a hemocytometer and counting the cells under a microscope.

Analysis of IL-17+ and IFN-γ⁺ cells in BAL. Cells were isolated as described in “Inflammatory cell population analysis in BAL” and then subjected to stain with anti-IL-17 and IFN-γ Ab based on the conventional methods for intracellular flow cytometry analysis. After staining, the change of IL-17+ and IFN-γ⁺ cells in BAL after treatment of alloferon were examined by flow cytometry analysis. Analysis of the expression of IL-6, TNF-α, and MCP-1, MIP-1, and MIP-2. Lung specimens were collected at 3 days after treatment of alloferon on bleomycin-treated mice, and then the analysis of inflammatory cytokines was performed using specific each pair of primers having a sequence selected from the group consisting of SEQ ID NO:3 to SEQ ID NO:16 (IL-6: SEQ ID NO:3 and SEQ ID NO:4, IL-17A: SEQ ID NO:5 and SEQ ID NO:6, MCP-1: SEQ ID NO:7 and SEQ ID NO:8, MIP-1: SEQ ID NO:9 and SEQ ID NO:10, MIP-2: SEQ ID NO:11 and SEQ ID NO:12, CXCL10: SEQ ID NO:13 and SEQ ID NO:14, 18s rRNA: SEQ ID NO:15 and SEQ ID NO:16).

### EXAMPLE 2

### Results

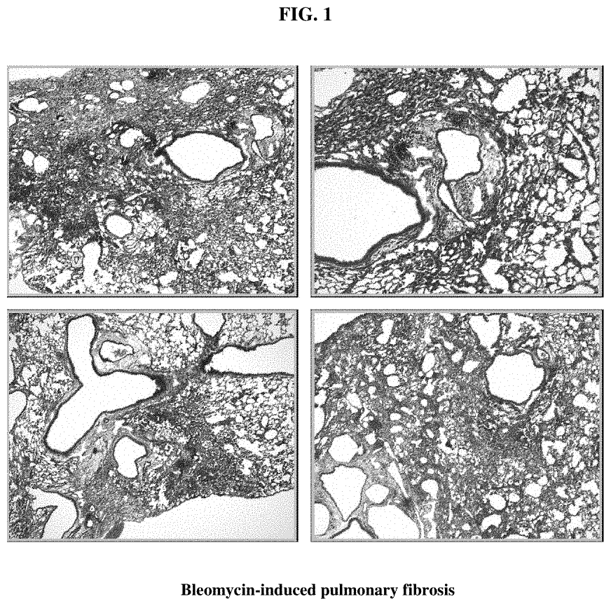

Bleomycin-induced pulmonary fibrosis induction. Bleomycin-induced lung fibrosis is a well-known animal model for human interstitial pulmonary fibrosis. The pathophysiology of interstitial fibrosis is characterized by repeated inflammation from known or unknown causes and reactive fibrosis. To test whether alloferon has some effects on these pathophysiologic processes, a bleomycin-induced lung fibrosis model was set up. C57BL/6 male mice were intratracheally inoculated with 1.5 mg/kg bleomycin in 55 μl volume, and the lungs of these mice were analyzed 21 days later. Lungs were fixed through intratracheal inflation of 4% paraformaldehyde solution and paraffin-embedded and stained with H&E and Masson's trichrome, the connective tissue staining (FIG. 1). Each figure was taken from different individuals of the control group (animals that were not administered alloferon). Bleomycin-treatment was seen to destroy the lung structures. Massive fibroblastic proliferation and mononuclear inflammatory cell infiltration was prominent in the pulmonary interstitial tissue. Collagen deposition characterized by blue color in Masson's trichrome staining was extensive and most densely around the main bronchi (FIG. 1).

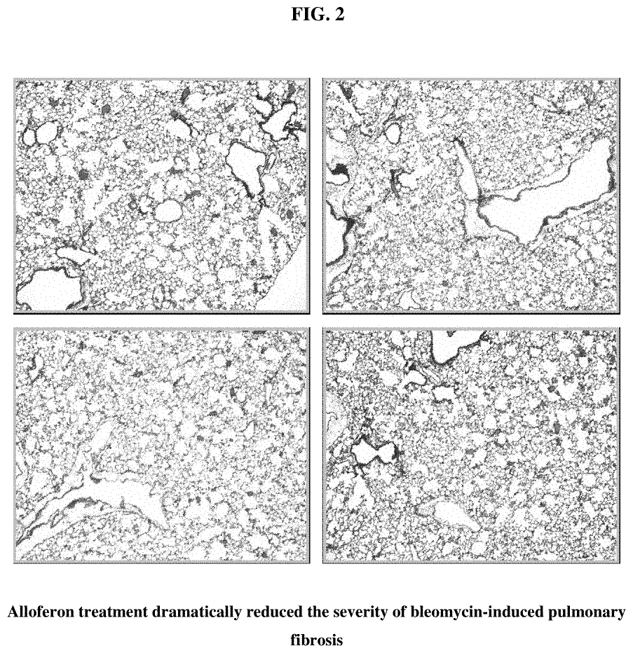

Alloferon treatment dramatically reduced the severity of bleomycin-induced pulmonary fibrosis. To evaluate the effects of alloferon on the pathogenesis of bleomycin-induced pulmonary fibrosis, 50 μg of alloferon was intraperitoneally injected every day into the mice from the same day of bleomycin-inoculation. Twenty-one days later, lungs of the mice were fixed through intratracheal inflation of 4% paraformaldehyde solution and paraffin-embedded and stained with H&E and Masson's trichrome. After treatment with alloferon, lung structures were almost normal with scant inflammatory cell infiltration and fibroblast proliferation. Airway spaces and structures were well-preserved, and some collagen deposits were noted around the main bronchi (FIG. 2). Each figure was taken from different individuals of the alloferon-treated group.

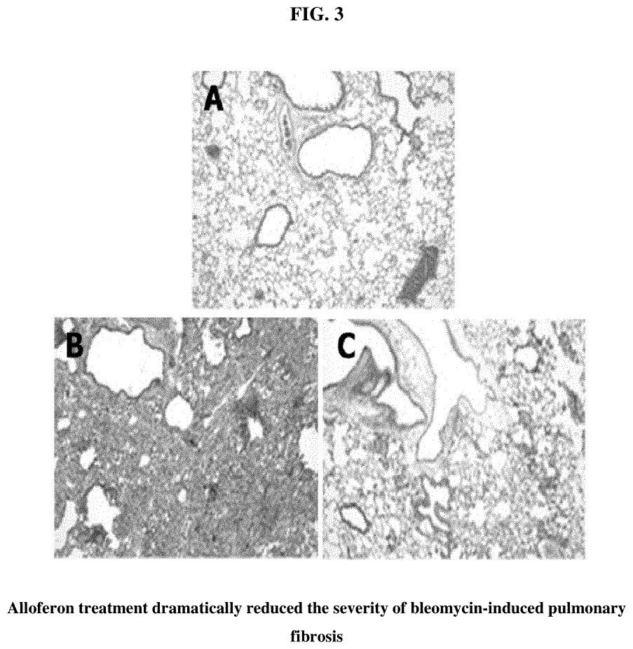

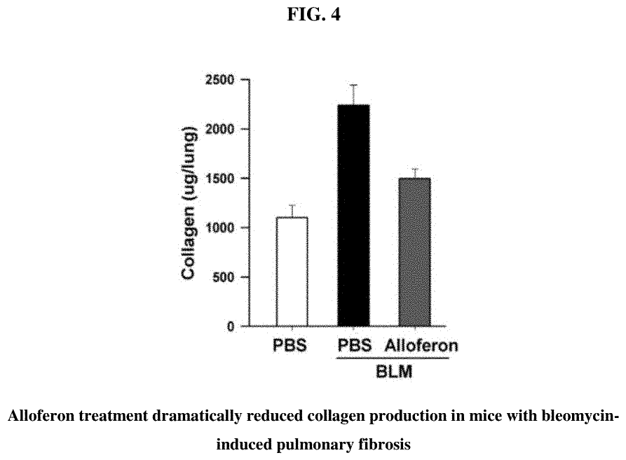

Collagen is the most abundant insoluble protein found in the extracellular matrix and connective tissues. It can be found in skin, tendons, bone, cartilage, muscle, vitreous humor and ligaments, among other tissues. The total content of collagen in fibrotic lung were measured to evaluate anti-fibrotic effect of alloferon in the bleomycin-induced pulmonary fibrosis model because the hyperproliferation of fibroblasts and increased collagen production is the irreversible severe event at the terminal stage of pulmonary fibrosis. As shown in FIG. 3, bleomycin increases collagen contents in the lung, but it is completely suppressed by the treatment of alloferon (FIGS. 3 and 4). The treatment of alloferon in animals shown in FIGS. 3 and 4 is the same as shown in FIG. 2. A treatment of 50 μg of alloferon was intraperitoneally injected every day into the mice from the same day of bleomycin-inoculation. 21 days later, and the lungs of the mice were fixed through intratracheal inflation of 4% paraformaldehyde solution and paraffin-embedded and stained with H&E and Masson's trichrome. FIG. 3 shows histology from (A) Control mice, (B) Mice with bleomycin, (C) Mice with bleomycin and alloferon. The fixation and staining shown in FIG. 3 were treated same as shown in FIGS. 1 and 2.

As shown in FIG. 4, alloferon treatment dramatically reduced collagen production in mice with bleomycin-induced pulmonary fibrosis. 50 μg of alloferon was intraperitoneally injected every day into the mice from the same day of bleomycin-inoculation. 21 days later, the lungs of the mice were collected, and the collagen content was measured. FIG. 4 shows the collagen content for (A) Control mice, (B) Mice with bleomycin, (C) Mice with bleomycin and alloferon.

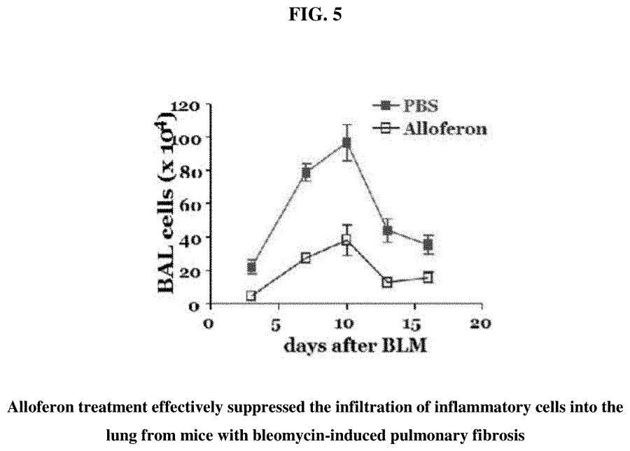

Alloferon treatment effectively suppressed the infiltration of inflammatory cells into the lung of bleomycin-induced pulmonary fibrosis. BAL is a simple and typical method commonly performed to diagnose pulmonary diseases. BAL is used to sample pulmonary components to determine immune cells in the lung. Pulmonary chronic inflammation plays a critical role in lung cancer initiation and progression. To clarify the underlying mechanism of inflammation, BAL cell counting in inflamed lung tissue from mice was used to determine the pulmonary immune response. 50 μg of alloferon was intraperitoneally injected daily into mice the same day of inoculation with bleomycin. Twenty-one days later, the recovered lavage fluid was collected in 1.5 ml Eppendorf tubes on ice. The BAL cell pellets were resuspended in 400 μl of PBS, and the cells were counted by using 20 μl of the cell sample in a hemocytometer and counting the cells under a microscope. As shown in FIGS. 1-3, alloferon effectively prevented the induction of severe bleomycin-induced pulmonary fibrosis, the infiltration of inflammatory cells into the inflamed lung was also investigated. Inflammatory cells in BAL fluid was effectively suppressed by the treatment of mice with alloferon (FIG. 5).

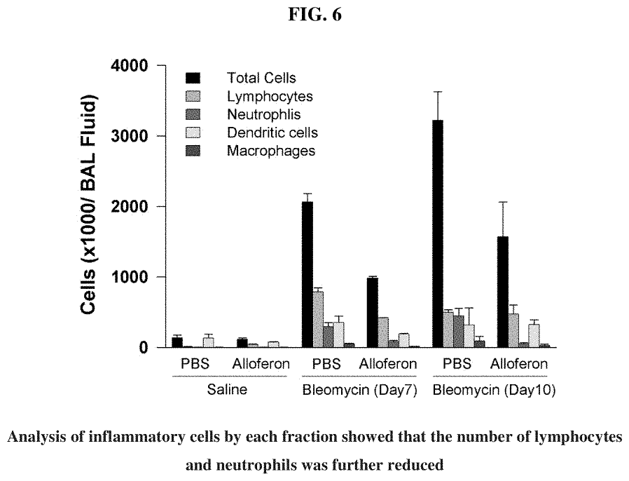

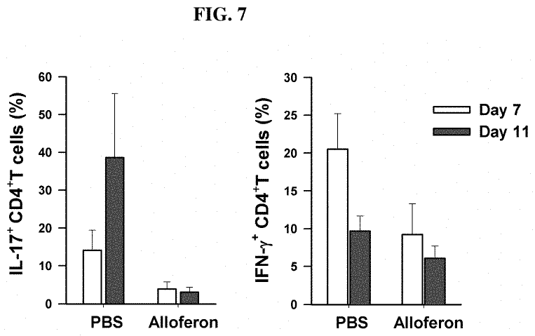

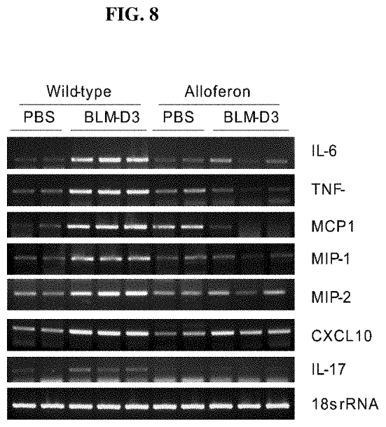

Infiltration of inflammatory cells and degree of fibrosis were significantly decreased in the group injected with 50 μg/ea alloferon daily. The number of inflammatory cells in BAL fluid at 7 and 10 days after injection of BLM showed that inflammatory cells were reduced by more than 50% in the group injected with alloferon compared to the control group injected with PBS (FIG. 6). Analysis of inflammatory cells by each fraction showed that the number of lymphocytes and neutrophils was further reduced. The intracellular staining of CD4+ T cells in BAL fluid at 7 and 11 days after BLM injection showed that IL-17+ and IFN-γ⁺ cells were significantly increased in alloferon injected group compared to PBS-treated control group (FIG. 7). It was confirmed that the expression of IL-6, TNF-α, and MCP-1, MIP-1, and MIP-2, which are inflammatory cytokines, was decreased in alloferon injected group (FIG. 8).

### CONCLUSION

The aforementioned description of the specific embodiments will so fully reveal the general nature of the disclosure that others can, by applying knowledge within the skill of the art, readily modify and/or adapt for various applications such specific embodiments, without undue experimentation, and without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

What is claimed is:

1. A pharmaceutical composition for treating pulmonary fibrosis comprising a compound of formula (I), X₁-His-Gly-X₂₋His-Gly-Val-X₃, wherein X₁ is absent or represents at least one amino acid residue, X₂ is a peptide bond or represents at least one amino acid residue, and X₃ is absent or represents at least one amino acid residue, or a physiologically acceptable salt thereof.

2. The composition according to claim 1, wherein the compound of formula (I) has an amino acid sequence of SEQ ID NO:1 or SEQ ID NO:2.

3. The composition according to claim 1, which suppresses the deposition of collagen.

4. The composition according to claim 1, which suppresses the infiltration of inflammatory cells.

5. A pharmaceutical formulation for treating pulmonary fibrosis comprising the composition of claim 1.


SEQUENCE LISTING (16 sequences)

SEQ ID NO: 1 - protein, 39 aa, Artificial Sequence
  alloferon
HISGLYVALSERGLYHISGLYGLNHISGLYVALHISGLY

SEQ ID NO: 2 - protein, 36 aa, Artificial Sequence
  alloferon
GLYVALSERGLYHISGLYGLNHISGLYVALHISGLY

SEQ ID NO: 3 - DNA, 23 bp, Artificial Sequence
  IL-6 primer 1
gaggataccactcccaacagacc

SEQ ID NO: 4 - DNA, 24 bp, Artificial Sequence
  IL-6 primer 2
aagtgcatcatcgttgttcataca

SEQ ID NO: 5 - DNA, 20 bp, Artificial Sequence
  IL-17A primer 1
tccagaaggccctcagacta

SEQ ID NO: 6 - DNA, 20 bp, Artificial Sequence
  IL-17A primer 2
acacccaccagcatcttctc

SEQ ID NO: 7 - DNA, 20 bp, Artificial Sequence
  MCP-1 primer 1
accacagtccatgccatcac

SEQ ID NO: 8 - DNA, 20 bp, Artificial Sequence
  MCP-1 primer 2
ttgaggtggttgtggaaaag

SEQ ID NO: 9 - DNA, 29 bp, Artificial Sequence
  MIP-1 primer 1
atgaaggtctccaccatgcccttgctgtt

SEQ ID NO: 10 - DNA, 30 bp, Artificial Sequence
  MIP-1 primer 2
gtctacctagaatagctgtcaccaaacagt

SEQ ID NO: 11 - DNA, 21 bp, Artificial Sequence
  MIP-2 primer 1
ccaagggttgacttcaagaac

SEQ ID NO: 12 - DNA, 25 bp, Artificial Sequence
  MIP-2 primer 2
tgaggtctttgagggatttgtagtg

SEQ ID NO: 13 - DNA, 24 bp, Artificial Sequence
  CXCL10 primer 1
ggaaccctagtgataaggaatgca

SEQ ID NO: 14 - DNA, 25 bp, Artificial Sequence
  CXCL10 primer 2
tgaggtctttgagggatttgtagtg

SEQ ID NO: 15 - DNA, 20 bp, Artificial Sequence
  18s rRNA primer 1
gtaacccgttgaaccccatt

SEQ ID NO: 16 - DNA, 20 bp, Artificial Sequence
  18s rRNA primer 2
ccatccaatcggtagtagcg